(19) United States
(12) Patent Application Publication
(10) Pub. No.: US 2012/0323320 A1
(43) Pub. Date: Dec. 20, 2012

(54) ADJUSTABLE INTRAOCULAR LENS

(75) Inventors: Aleksey Nikolaevich Simonov, DV Den Haag (NL); Michiel Christiaan Rombach, BA Breda (NL)

(73) Assignee: AKKOLENS INTERNATIONAL B.V., Breda (NL)

(21) Appl. No.: 13/512,152

(22) PCT Filed: Nov. 30, 2010
(86) PCT No.: PCT/NL2010/050803

§ 371 Date: Sep. 6, 2012

(30) Foreign Application Priority Data
Nov. 30, 2009 (NL) 2003881

(51) Int. Cl. A61F 2/16

(52) U.S. Cl. 623/6.22

(57) ABSTRACT

An adjustable intraocular lens is disclosed comprising at least one optical element and at least one haptic. Rotation of the lens in a plane perpendicular to the optical axis and bounded by an oval boundary adjusts the lens diameter which provides a corresponding adjustment of optical power. Preferably the lens according to the invention is adapted to be manipulated by manipulation means outside the eye, for example, surgical means. Adjustment of the angular position of the intraocular lens results in a corresponding adjustment of the lens diameter which, in turn, leads to a corresponding adjustment of the optical power of the lens.

The intraocular lenses (IOL) are intended to replace the natural lens of the eye which, for example, has become opaque due to cataracts. In many cases, however, the prescribed, i.e. calculated, optical power (equivalently, the focusing power) of the lens does not exactly match the actual optical power that is required for a particular eye to provide sharp distant vision, i.e. emmetropia.

The present document discloses intraocular lenses (IOLs) with adjustable optical power that can be adjusted, for example, during the surgery, or after the surgery in which the lens is implanted. These adjustable lenses can be used to correct the refractive error of the eye.

The adjustable intraocular lenses, described in the present document, comprise at least one optical arrangement for adjusting the optical power which optical arrangement includes at least one optical element.

The lens also comprises at least one haptic, being positioning means, to position the lens in the eye, in contact with natural components of the eye, for example, in contact with the sulcus, or in contact with an ocular ring, a supporting device for the lens. The sulcus is a preferred position for the lens because the sulcus diameter generally varies depending on the angular direction in the plane of the sulcus, a plane required for functioning of the adjustable lens. So, the shape of the sulcus resembles an oval which is essential for the first embodiment of the adjustable lens.

The optical arrangement and haptics of the lens are adapted to provide adjustment of the optical power by adjusting the angular position of the lens. The adjustment of the angular position of the lens can be made by rotation, i.e. turning the lens by external means, for example, by a surgical needle, in a plane perpendicular to the optical axis of the eye. The plane of rotation is generally perpendicular to the optical axis of the eye and is bounded (from the inner side) by a contour with a diameter depending on the angular direction, in the plane of rotation. For example, the contour can have an oval shape and coincide with the natural shape of the sulcus.

External means are the manipulation means outside the eye, for example, surgical means. Neither the ciliary muscle of the eye nor any other forces in the eye should affect angular position of the lens. So, the adjustment of the angular position of the intraocular lens results in a corresponding adjustment of the lens diameter which, in turn, leads to a corresponding adjustment of the lens optical power.

The lens can be designed such that the adjustment of its diameter provides a corresponding adjustment of an axial position of at least one optical element and that the adjustment of axial position, in turn, corresponds to adjustment of optical power of the lens. The axial position denotes the element position along, or parallel to, the optical axis of the eye. In such a design a single axially moving lens as described, for example, in DE60225439, or, alternatively, multiple lenses as disclosed, for example, in WO2005104995, a telescope design with two lenses moving along the optical axis which can provide the desired adjustment of optical power.

Alternatively, the lens can be designed such that the adjustment of diameter is translated into a corresponding adjustment of lateral position of at least one optical element which adjustment, in turn, corresponds to a corresponding adjustment of optical power of the lens. Lateral position means position of at least one optical element in a plane perpendicular to the optical axis of the eye. In such design, for example, multiple optical elements described in EPA1720489, WOA 2007015640, WOA2006118452 and WOA2007027091, with two free-form optical elements shifting perpendicular to the optical axis, can provide the desired adjustment of optical power.

Alternatively, the lens can be designed such that the adjustment of its diameter is translated into a corresponding adjustment of the radius, i.e. radius of curvature, of at least one surface of at least one optical element which adjustment, in turn, corresponds to a corresponding adjustment of the optical power of the lens. Adjustment in radius generally means bulging of the pliable material of at least one optical element under a force largely in the direction of any axis in a plane perpendicular to the optical axis of the eye. In such design, for example, a lens with the optomechanical concept according to WO0067678 and DE60313846, with a single lens of variable radius, can provide the desired adjustment of optical power.

In the aphakic eye, an eye with the removed natural lens, a fixed (not depending on the state of the eye) refractive correction is required to provide sharp vision. Therefore, an intraocular lens which replaces the natural lens must provide a fixed optical power, which power is generally in the range of 15-20 diopters (D). The lenses disclosed in the present document can be adapted to provide such fixed optical power. A perfect in situ adjustment of this fixed refractive optical power will result in a perfect emmetrope eye, being an eye with a focus at infinity. In many cases, however, emmetropia of the eye is not obtained that perfectly and a residual refractive error of, say, 0.5-2 D can remain, for example, an error due to optometric measuring errors, or, an error developing over time, due to natural changes in the eye, or, an error, during implant surgery, due to a settling plane of the lens along the optical axis which differs from the expected plane. The adjustable lenses disclosed in the present document aim to adjust for such residual refractive errors. Such adjustments can be expected to occur only occasionally, for example once during surgery, or, for example, only once or twice after implantation.

The adjustable intraocular lens can be designed for different functions to be positioned in different locations in the eye. Firstly, in the anterior chamber of the eye, as a phakic lens which functions in combination with, for example, the natural lens and of which minor refractive errors can be corrected even long after implantation. Such lens comprises haptics adapted to position the lens in anterior chamber of the eye. The adjustable intraocular lenses disclosed in this document can be combined with existing phakic intraocular optics.

Secondly, in the posterior chamber of the eye, as an aphakic lens that replaces the natural lens. Such an aphakic lens can be positioned in the capsular bag, in the lens plane, and comprise haptics adapted to position the lens in the capsular bag. Alternatively, such aphakic lens can also be positioned in the sulcus, at the sulcal plane, and comprise haptics to position the lens in the sulcus. The adjustable intraocular lenses disclosed in this document can be combined existing aphakic intraocular optics such as monofocal intraocular optics (providing a single fixed focus), multifocal intraocular optics (providing multiple fixed foci) and accommodating intraocular optics (optics providing variable focus).

In a lens adapted to be positioned in the sulcus the haptics can comprise at least one anterior flange adapted for placement in the sulcus, or, alternatively, the haptics can comprise at least two flanges, at least one anterior flange and at least one posterior flange which combination of flanges is adapted to embrace the ciliary mass. Flanges can be fitted with fixation components such as undulations or hooks to prevent undesired rotation, by unintended movements in the eye, and still allow desired rotation, by intended external force.

The lens can be included in a lens combination which combination also includes an ocular ring, being an additional component, separate from the lens. The ring is positioned in the eye, for example, in the capsular bag, or in the sulcus, and the lens is, in turn, placed inside the ring. Such a ring comprises a contour/bound with a diameter depending on the angular direction, for example, an oval contour. The plane of the contour is perpendicular to the optical axis. The combination of a lens and a ring is adapted to provide adjustment of the optical power of the lens by corresponding adjustment of the angular position of the lens in the ocular ring, by turning the lens in the ring in the plane perpendicular to the optical axis. Such ring might be required because not all the components of the eye offer the required bounds/contour with a diameter depending on the angular direction. For example, the capsular bag is largely round, not oval, and, for example, not all sulci in all eyes have the required degree of ovality for the adjustment to function. In addition, the degree ellipticity, i.e. the ratio between the largest and smallest diameters of the ocular ring, is highly predictable and can be optimized by proper design and choice of material, which criteria generally do not apply to components of the eye. So, the lens is included in a lens combination which combination also includes an ocular ring inside which the lens is positioned and which ocular ring comprises a contour/bound with a diameter depending on the angular direction. The optical power of the lens can be adjusted by rotation of the lens with respect to the ocular ring. During rotation the lens, for example, displaces in the plane perpendicular to the optical axis, or, alternatively, it moves along the optical axis, or, alternatively, the lens experiences mechanical deformations resulting in changing of the optical power.

The lens can comprise at least one flange adapted for positioning in the ocular ring and which flange holds the lens in the ocular ring such that the lens can only be rotated by intended external means and manipulations and not by unintended movements of the eye.

The ring can be an anterior chamber ocular ring adapted to position the combination in the anterior chamber of the eye and comprising, for example, specific fixation components adapted to position the ring in the anterior chamber. Alternatively, the ring can be a capsular bag ocular ring to position the combination in the capsular bag of the eye and comprising, for example, specific fixation components adapted to position the ring in the capsular bag. Alternatively, the ring can be a sulcus ocular ring adapted to position the combination in the sulcus of the eye and comprising, for example, fixation components adapted to position the ring in the sulcus. Also, the ring construction with the oval internal bound can provide additional adaptations to optimize function of the lens, for example, stepwise changes in diameter, for example, complementary undulations, steps/teeth, corresponding to the undulations on the flange of the lens which allow precise and stepwise adjustments of optical power of the lens.

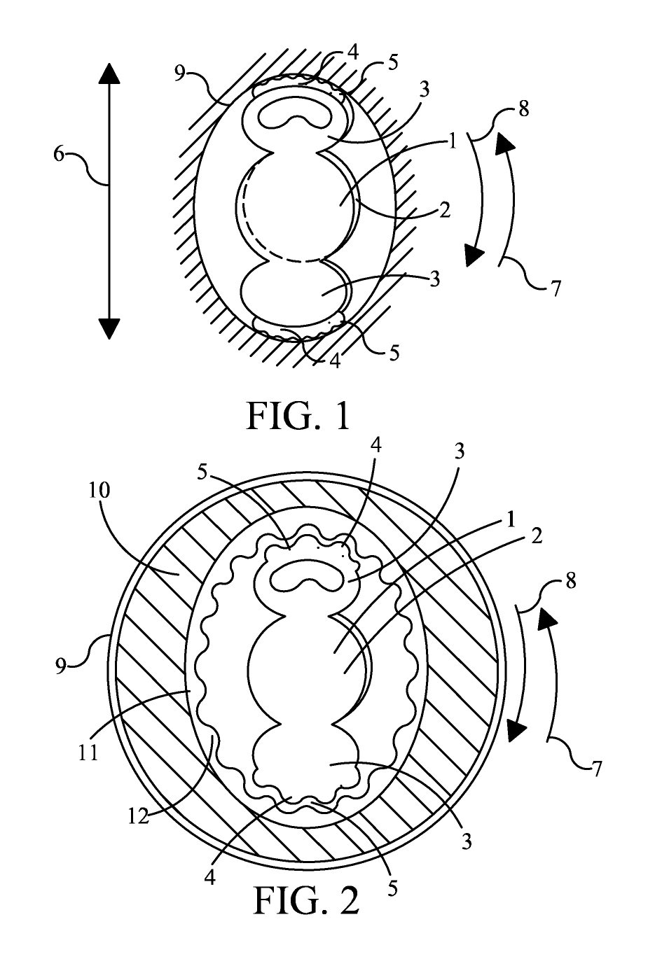

Subsequently the present invention will be elucidated with the help of the accompanying drawings wherein FIGS. 1 and 2 disclose lenses with laterally shifting optical arrangements and optical elements with largely cubic surfaces according to WO2009051477 are used to illustrate the present invention.

FIG. 1 shows an embodiment of an adjustable intraocular lens, with optical arrangement according to WO2009051477. Lens with anterior, 1, and posterior, 2, optical element, connecting component, 3, flange, 4, in this example fitted with undulations, 5, for improved sulcus fixation, which lens can be rotated, in this example, counter-clockwise, 7, or clockwise, 8, to a smaller sulcus diameter to increase optical power. (This example concerns a vertical starting position of the lens: a decreased optical power caused by rotation in any direction results from a start at such horizontal starting position. In other starting positions, the increase or decrease of the optical power of the lens can be obtained). The flanges position the lens in the sulcus, 9 and the undulations on the flanges prevent undesired rotation of the lens.

FIG. 2 shows an alternative embodiment combination of a lens and an ocular sulcus ring. (For details of lens and concepts of rotation see FIG. 1) A ring, 10, in this example, with circular outside shape, 9, and an oval inside shape, 11, with inside shape fitted with undulations, 12, to lock undulations on the flange of the lens, 5, allowing rotation by external force only.

A method is required to adjust the optical power of the lens. The lens is rotated by external means, in the plane bounded by a contour with a diameter depending on the angular direction. The plane is generally perpendicular to the optical axis of the eye and may be positioned, for example, in the sulcus of the eye. Alternatively, to adjust the optical power, the lens can be rotated in an ocular ring.

External means and manipulations include any intended means and manipulations from outside the eye, for example movement by a surgical tool, and exclude unintended actions from inside the eye, for example movement of the ciliary muscle. The rotation of the lens can be achieved by, invasive, surgical means, for example manipulation by surgical tools, for example a needle or hook, via a small paracentesis, during minor eye surgery in an out-patient procedure. Alternatively, a non-invasive, means can be used, for example, magnetic means, with, at least one, magnet as a component of the lens construction in combination with an external magnet. Alternatively, other non-invasive means can be employed, for example, laser light means, which includes a heat source, for example, at least one ophthalmological laser and at least one thermal deformable component of the lens, for example, comprising a bimetallic element. These means for adjustment are examples and means for adjustments are not restricted hereto.

The combination of lens and method disclosed in the present document provide correction of residual refractive error of the eye.

The present invention is not restricted to the optical arrangements described above and may include alternatives.

1. An adjustable intraocular lens comprising at least one optical arrangement for adjustment of optical power which arrangement comprises at least one optical element and at least one haptic to position the lens in the eye, the lens adjustable by external means only, wherein the lens is adapted to provide adjustment of optical power by adjustment of the angular position of the lens in the plane bounded by a contour with a diameter depending on the angular direction.

2. The lens according to claim 1, wherein the adjustment of rotational angle provides a corresponding adjustment of diameter of the lens.

3. The lens according to claim 1, wherein the adjustment of diameter provides a corresponding adjustment of optical power of the lens.

4. The lens according to claim 1, wherein the adjustment of diameter provides a corresponding adjustment of axial position of at least one optical element of the lens which adjustment of axial position provides, in turn, a corresponding adjustment of optical power of the lens.

5. The lens according to claim 1, wherein the adjustment of diameter provides a corresponding adjustment of lateral position of at least one optical element which adjustment of lateral position provides, in turn, a corresponding adjustment of optical power of the lens.

6. The lens according to claim 1, wherein the adjustment in diameter provides a corresponding adjustment of at least one radius of at least one surface of at least one optical element which adjustment of radius provides, in turn, a corresponding adjustment of optical power of the lens.

7. The lens according to claim 1, wherein the haptics are adapted to position the lens in the anterior chamber of the eye.

8. The lens according to claim 1, wherein the haptics are adapted position the lens in the posterior chamber of the eye.

9. The lens according to claim 8, wherein the haptics are adapted to position the lens in the capsular bag.

10. The lens according to claim 8, wherein the haptics are adapted to position the lens in the sulcus.

11. The lens according to claim 10, wherein the haptics comprise at least one anterior flange which flange is adapted for placement in the sulcus.

12. The lens according to claim 10, wherein the lens comprises a combination of flanges which combination includes at least one anterior flange and at least one posterior flange which combination is adapted to embrace the ciliary mass.

13. The lens according to claim 1, wherein the lens is included in a lens combination which combination also includes an ocular ring and which combination is adapted to provide adjustment of optical power of the lens by corresponding adjustment of the rotational angle of the lens in the ocular ring.

14. The lens according to claim 13, wherein the lens comprises at least one flange adapted to provide a positioning of the lens in the ocular ring such that the lens can be rotated by external means only.

15. The lens combination according to claim 13, wherein that the ring is an anterior chamber ocular ring adapted to position the combination in the anterior chamber of the eye.

16. The lens combination according to claim 13, wherein the ring is a capsular bag ocular ring adapted to position the combination in the capsular bag.

17. The lens combination according to claim 13, wherein the ring is a sulcus ocular ring adapted to position the combination in the sulcus.

18. The lens according to claim 1, wherein the lens is an adjustable phakic intraocular lens.

19. The lens according to claim 1, wherein the lens is an adjustable aphakic intraocular lens.

20. The lens according to claim 1, wherein the lens is an adjustable aphakic accommodating intraocular lens.

21. A method to adjust an adjustable intraocular lens according to claim 1, wherein the method includes rotating the lens in the plane bounded by a contour with a diameter depending on the angular direction.

22. The method according to claim 21, wherein the method includes rotating the lens by surgical means.

23. The method according to claim 21, wherein the method includes rotating the lens by magnetic means.

24. The method according to claim 21, wherein the method includes rotating the lens by laser light means.

25. The lens according to claim 1, wherein the lens is adapted to provide correction of residual refractive error of the eye.
